(19) United States
(12) Patent Application Publication
(10) Pub. No.: US 2024/0390007 A1
(43) Pub. Date: Nov. 28, 2024

(54) Implantable Drug Eluting System and Method of Use

(71) Applicant: P Tech, LLC, Manalapan, FL (US)

(72) Inventor: Peter M. Bonutti, Manalapan, FL (US)

(21) Appl. No.: 18/651,323

(22) Filed: Apr. 30, 2024

Related U.S. Application Data
(63) Continuation of application No. 17/102,125, filed on Nov. 23, 2020, now abandoned.
Application No. 17/102,125 is a continuation of application No. 15/726,599, filed on Oct. 6, 2017.
Application No. 15/726,599 is a continuation of application No. 11/842,648, filed on Aug. 21, 2007, now abandoned.
(60) Provisional application No. 60/822,966, filed on Aug. 21, 2006.

(51) Int. Cl. A61B 17/12; A61B 17/00; A61B 17/132; A61B 17/135; A61B 17/70; A61B 17/72; A61B 17/88; A61B 18/00; A61B 18/14; A61F 2/38; A61F 2/44; A61K 31/215; A61K 31/715; A61K 35/24; A61K 38/48; A61L 27/22; A61L 27/52; A61L 31/04; A61L 31/14

(52) U.S. Cl. CPC A61B 17/12; A61B 17/12136; A61B 17/1325; A61B 17/70; A61B 17/72; A61K 31/215; A61K 31/715; A61K 35/24; A61K 38/4833; A61L 27/222; A61L 27/52; A61L 31/045; A61L 31/145; A61B 2017/005; A61B 2017/00893; A61B 2017/12004; A61B 17/135; A61B 17/8847; A61B 2018/00011; A61B 2018/00023; A61B 2018/0022; A61B 18/14; A61F 2/38; A61F 2/44; A61L 2400/04

(57) ABSTRACT

The present disclosure provides a system for injecting a drug eluting construct in a patient. The construct includes multiple cellular based microcaspules, wherein the multiple cellular based microcapsules create at least one of a plurality of layers or sections of microcapsules joined together to comprise an implant. There is a medicinal agent within the microcapsules. A syringe and needle inject the implant constructed of microcapsules into the patient at least one of during and after a surgical procedure. The medicinal agent controllably releases into the patient both immediately and at a delayed time.

### CROSS-REFERENCE TO RELATED APPLICATIONS

This application is a continuation of U.S. Non-Provisional patent application Ser. No. 17/102,125 filed Nov. 23, 2020, entitled IMPLANTABLE DRUG ELUTING SYSTEM AND METHOD OF USE, which is a continuation of U.S. Non-Provisional patent application Ser. No. 15/726,599, filed Oct. 6, 2017, entitled IMPLANTABLE DRUG ELUTING SYSTEM AND METHOD OF USE, which is a continuation of U.S. Non-Provisional patent application Ser. No. 11/842,648, filed Aug. 21, 2007, entitled METHOD OF INHIBITING THE FORMATION OF ADHESIONS AND SCAR TISSUE AND REDUCING BLOOD LOSS, which claims the benefit of U.S. Provisional Patent Application 60/822,966 to the same inventor, filed Aug. 21, 2006, entitled METHOD OF INHIBITING THE FORMATION OF ADHESIONS AND SCAR TISSUE, the entire contents of which are incorporated herein by reference.

### FIELD OF THE INVENTION

The present invention relates to a method of reducing blood loss during surgery, while inhibiting the postoperative formation of fibrosis, and more particular for inhibiting scar formation and surgical adhesions, as well as decreasing exogenous bone formation.

### BACKGROUND OF THE INVENTION

The importance of reducing blood loss during surgery is well understood. In the prior art, bandages are applied with pressure to reduce bleeding, and cold is applied to reduce edema. To address adhesion formation, barrier films are applied between abraded or cut tissue.

Devices to cool postoperatively include the dental ice pack of U.S. Pat. No. 6,217,606 to Portnoy et al., the dental compress of U.S. Pat. No. 4,983,122 to Mitnick, and the fluid circulating device of U.S. Pat. No. 5,190,032 to Zacoi. The dental devices enclose a gel in a flexible envelope. The device is cooled and positioned adjacent the surgical site postoperatively. The fluid circulating device is intended to be more easily positioned adjacent to the surgical site than prior art devices. While these devices address postoperative blood loss and edema, as well as patient comfort, they are not directed to blood loss during surgery. More particularly, they are not adapted to be used during surgery, as they would operate to entirely obscure the operating field.

U.S. Pat. No. 3,867,939 to Moore discloses an absorbent fluid circulating dressing or surgical sponge designed to be used in an open wound. The device includes connections for a cold water supply and return, and circulates the fluid between layers of paper, scrim, and plastic film. While perhaps more compact than the chemical cooling packs of the prior art, discussed therein, the device none the less imposes considerable bulk in the context of insertion into an active surgical field. In addition, there are logistical problems of supplying cooled water, as well as having available an adequate supply in the correct sizes. Further, designed as a disposable device, the apparatus of Moore introduces considerable cost.

An additional problem with Moore, and other devices which introduce preformed panels or dressings, such as U.S. Pat. No. 5,409,472 to Rawlings, is that it is difficult or impossible to cover the entire portion of the operating field that is bleeding, while leaving the area of interest completely unobscured and unobstructed.

The formation of scar tissue is a normal sequel to surgery or other tissue injury and is required for proper wound healing. In some cases, however, the scar tissue overgrows the intended region and creates surgical adhesions. These scar tissue surgical adhesions restrict the normal mobility and function of affected body parts. Where peripheral nerves are involved, fibrous adhesions can elicit severe pain during normal movement. Furthermore scars and keloid tissue (raised scar tissue) are often unsightly and present psychological and emotional problems.

Therefore there exists a need to not only reduce blood loss during and after surgery, but also to reduce postoperative adhesions. There are various approaches to reducing adhesions, but none of them solve the problems described above with respect to blood loss during surgery.

In particular, U.S. Pat. No. 5,711,958 to Cohn et al., incorporated by reference herein, discloses bioabsorbable polymeric materials which were found to inhibit the formation of adhesions, administered as rods, cylinders, foams, dispersions, viscous solutions, liquid polymers, sprays or gels. An example provided includes using a 10 mil thick film sutured into rabbits having abraded intestines and removed muscle. There are no other examples provided for using the other polymer forms mentioned, and there is no suggestion as to how the film employed might be used to reduce bleeding.

U.S. Pat. No. 6,607,512 to Oliver, et al, incorporated herein by reference, discloses a device for delivering an anti-adhesion gel during surgery, including applying a gel in both endoscopic and open incision procedures. The device disclosed allows the surgeon to apply the gel as one would apply paint with a paint brush, in an even layer.

In addition to formation in connection with abrasions, as discussed with respect to Cohn, above, adhesions also form in association with implants. This is addressed in U.S. Pat. No. 6,187,043 to Ledergerber, which discloses coating implants, particularly breast implants, with filaments of expanded PTFE (PTFEe). Woven PTFEe is attached to a fabric backing, which is used to encapsulate the implant.

A variety of anti-adhesion compositions are known, and are disclosed in the cited references, as well as in U.S. Pat. No. 6,869,938 to Schwartz et al. In Schwartz, such compositions are incorporated into membranes, sponges, and microspheres. Schwartz discloses that sponges can be useful for hemostasis, but provides no further details as to how the sponges might be used. U.S. Pat. No. 5,176,700 to Brown discloses a laparoscopic intra-abdominal device for blunt manipulation of a sponge including direct hemostasis pressure on small blood vessels.

Thus various approaches to the reduction of blood loss during surgery are disclosed, including localized cooling, but they obscure and obstruct the operating field. Various solutions to the problems attendant to adhesion formation are disclosed, but they do not incorporate a solution to blood loss during surgery. It is therefore an object of the invention to provide an integrated solution to the problems of blood loss and adhesion, while avoiding the limitations of the prior art.

### SUMMARY OF THE INVENTION

The present invention provides for the reduction of blood loss during surgery, as well as the reduction in the formation of postoperative adhesions. Other advantages are realizable in connection with the apparatus and methods of the invention, as further described below.

In accordance with one embodiment of the invention, a surgical field is filled with a viscous substance, such as gelatin or a polymeric solution, which serves to retard or block the ingress of blood from surrounding tissue. The viscous substance may be optically clear, or may introduce some optical distortion, however some visualization of the surgical field remains, whereupon surgery may take place through the viscous substance.

Alternatively the bulk of the viscous substance immediately surrounding the area of interest may be removed, as by manipulation and or irrigation, so that an unobscured field of view, and unobstructed target area is realized. Gel at the periphery of the field is left intact, to continue to retard entry of blood into the operating field.

The aforedescribed process may be used in both endoscopic and open incision procedures. In accordance with another embodiment of the invention, the surgical field is sprayed with a cooling solution to cool the surrounding tissue sufficiently to achieve vasoconstriction, and thus reduce blood flow temporarily while the viscous substance is being applied. Depending upon the material used, the viscous substance may additionally be treated with heat, as by UV, RF, or warm air, or cooled, as by cool spray or cool air, in order to be cured or hardened and made more durable. Postoperatively, a heat cured or cold set dressing may be formed as described above, providing good support to healing tissue, and protection from infection.

The viscous substance may, in accordance with the invention, be formed to contain agents which aid healing or provide other therapeutic benefit, particularly substances which reduce the incidence of the formation of adhesions, which are discussed in greater detail below. Additional agents include blood clotting agents, non-steroidal anti-inflammatories, steroidal agents, analgesics, morphine, lidocaine, other anesthetics, calcium, thrombin, hyaluronic acid, and epinephrine.

In accordance with a further embodiment of the invention, the introduction of a viscous substance produces a distinct advantage when working with surgical adhesives and cements, such as bone cement. When cement is applied, it is common for a quantity of cement to escape into surrounding tissue. Removal of this cement can be a time consuming process, and introduces additional risk, particularly during kyphoplasty and other work proximate delicate tissue. When the surrounding tissue is coated with a viscous substance, particularly gelatin or lubricants, the removal of cement is greatly facilitated.

As described above, the immediate surgical field may be cooled to promote vasoconstriction, and thus reduce blood flow and blood loss. In accordance with the invention, all or a portion of a limb may be partially or completely surrounded by a cuff which is operative to squeeze the limb, and thus act as a tourniquet, while simultaneously cooling blood flowing into the limb. Accordingly, the degree to which the limb must be squeezed may be reduced when combined with cooling, and thus the trauma to muscle tissue is thereby reduced. The cuff is supplied with cold or hot air or liquid, so that the temperature may be adjusted by either a computer or the surgical practitioner during surgery. Temperature control in this manner is particularly useful for limb salvage surgery. The foregoing apparatus and method may advantageously be combined with epinephrine, marcaine, or other vasoconstrictive agent.

In accordance with another embodiment of the invention, a balloon is placed within a surgical field, operative to apply pressure to bleeding tissue. The balloon is inflated either before or after placement. Where the balloon is inserted before inflation, inflation pressure is advantageously used to distract, retract, or otherwise displace tissue. The balloon may be sized to span the entire surgical field, whereby pressure is applied to at least two sides of the field. Alternatively, the balloon may be wedged between tissue within the field, such as bone or soft tissue, and the bleeding tissue. Multiple balloons may be used.

In another embodiment of the invention, the balloon is caused to harden after inflation, whereupon portions of the balloon which are not engaged with bleeding tissue, and which are not needed for support, are excised. Where it is desired to leave the remaining balloon portion within the body for a period of time, the balloon may advantageously be fabricated with a biodegradable material. Hardening is accomplished by coating the balloon with a gel or polymer, as described above, which is set or cured by cooling or heating. In this manner, the gel or polymer is disposed proximate the bleeding tissue, and imparts the blood loss reduction benefits described above.

In one embodiment, heating elements are disposed on or within the balloon. Alternatively, heat or cold is created by disposing chemical heat or cold pack units within the balloon. Packs designed to generate heat or cold upon snapping or breaking a barrier between separated chemical components, as known in the art, are conveniently deployed within the balloon for this purpose. Balloons may additionally be provided with channels for conducting hot or cold liquids or gases.

Additionally, the balloons may be inflated and deflated during the surgical procedure, in order to gain access to different areas of the surgical field, or to restore compression to bleeding tissue. The compressive force is adjusted by varying the internal balloon pressure, or the force with which the balloon is wedged within the surgical field. In endoscopic procedures, balloons are inserted in a deflated state, and inflated once positioned. Advantageously, inflation pressure is only slightly higher than capillary pressure, whereby any burden on contacted tissue is minimized.

Balloons may be inflated with a gas or a liquid. Where the balloon is to be cut open, or is vulnerable to being pierced or broken, a biocompatible material, such as filtered air or sterile water, is of benefit.

In accordance with a further embodiment of the invention, retractors and other tools used within the surgical field are advantageously heated or cooled. As tools are commonly fabricated using metal, such tools may be heated or cooled prior to use. Alternatively, tools in accordance with the invention, having channels for the conduction of heated or cooled liquid or gas are advantageously deployed. Cooled tools contribute to vasoconstriction, and may additionally be coated with gelatin or polymer gels, with attendant benefits, as described above.

As described for tools, above, an implant may similarly be heated or cooled, as well as coated with gelatin or gels, as described above. Implants are similarly advantageously provided with channels for cooling or heating. In addition, implants are provided with means for generating heat once an implant is secured and sealed within the body. In this manner, postoperative pain is reduced, and healing accelerated. Heating may be accomplished by dielectric or induction heating, or other means not requiring an electrical connection.

A medical implant in accordance with the invention can be fabricated, for example, with biodegradable polymers, cellular based materials, or other biodegradable material. The implant may additionally include a plurality of layers, each including biologic agents as described herein. Each of the multiple layers may contain the same biological agent, or medicinal agents. A treatment protocol may require that different dosages of the medicinal agent or different composition of the medicinal agent be released at different times during the treatment protocol, an immediate release vs. a delayed/retarded release. Microcapsules containing the agent or medicament are additionally contemplated, either forming one or more layers, or forming the entire implant. Implants advantageously include bone spacers or other bone implants, where the formation of adhesions can be particularly problematic.

In accordance with another embodiment of the invention, the devices and methods described above may be combined with increasing the atmospheric pressure in the operating room, in the patient, or within the surgical field, thereby further reducing blood loss.

In accordance with yet another embodiment of the invention, a combination of therapeutic substances may be administered to the patient, cooperative with the devices and methods of the invention, to increase the overall efficacy of the procedure. These may be delivered before or after surgery, and may be timed release. Additionally, any implanted device, e.g. balloon or other implant, in accordance with the invention, may be formulated to be drug eluting, either through incorporation into the gelatin or gel matrix which coats the device, as described above, or by formulating the device to contain therapeutic substances which are released by known means, including biodegradation.

In accordance with a further embodiment of the invention, system or local pH is made more alkaline, in order to decrease the caustic effect of bleeding, thus protecting soft tissue and decreasing pain.

As discussed above, it is an object of the invention to reduce the formation of adhesions through introducing into the surgical field a biologic agent, to inhibit scar formation, in particular, surgical adhesions and exogenous bone formation. The biologic agent is biodegradable and is thus reabsorbed over a period of time. The biologic agent can be used to prevent or inhibit the formation of adhesions in an animal following any type of surgery or trauma, by applying an effective amount of the biologic agent to a wound site, through incorporation into a gelatin or gel matrix, applied directly or to an implant, or through incorporation into an implant, as described above.

The wound site refers to a site of tissue that has been injured in any manner, e.g., through surgery, contusion, abrasion and so forth, and also refers to tissue or organs that are adjacent to the injured tissue. For example, the biologic agent may be used to prevent or inhibit adhesions that form in relation to intestinal surgery, e.g., bowel resection, hernia repair, etc., which may cause obstruction of the intestine. The biologic agent may also prevent or inhibit adhesions or exogenous bone formation that can form near a bone fracture site, joint repair or replacement site, the formation of which may reduce or hinder the normal movement of the area of repair by restricting the natural movement of tendons over adjacent bone.

To aid in healing, the composition can additionally include a medicinal agent. Exemplary medicinal agents include drugs, enzymes, proteins, hormones, peptides, glycoproteins, or diagnostic agents such as releasable dyes which may have no biological activity per se.

Examples of classes of medicinal agents that can be used include antimicrobials, analgesics, antipyretics, anesthetics, antiepileptics, antihistamines, anti-inflammatories, anti-clotting agents, bone morphogenic proteins, cardiovascular drug, diagnostic agents, sympathomimetics, cholinomimetics, anti-muscarinics, antispasmodics, hormones, growth factors, muscle relaxants, adrenergic neuron blocks, anti-neoplastics, immunosuppressants, gastrointestinal drugs, diuretics, steroids and enzymes. It is also intended that combinations of medicinal agents can be used.

In addition to or as an alternative to, the medicinal agent may be a therapeutic agent. Examples of such agents include, but are not limited to, hormones, cells, fetal cells, stem cells, bone morphogenic proteins (BMPs), enzymes, proteins, RNA, germicides, gene therapy substances, cell therapy substances, viruses, etc.

In an embodiment the biologic agent is synovial fluid. The synovial fluid can be harvested from the patient prior to or during the surgical procedure by known techniques. Alternatively, the synovial fluid can be harvested from a donor.

Alternatively, the biologic agent is cerebrospinal fluid. The cerebrospinal fluid can be harvested from the patient prior to or during the procedure by known techniques. Alternatively, the cerebrospinal fluid can be harvested from a donor.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 is an illustration of an embodiment of the present disclosure, for example an operating field including anatomical elements of a body.

FIG. 2 is an additional illustration of an embodiment of FIG. 1, for example, including a substance in the operating field.

FIG. 2A is an additional illustration of an embodiment of FIG. 1, for example, including a substance positioned with respect to a blood vessel.

FIG. 3 is an additional illustration of an embodiment of FIG. 1, for example, after a portion of the substance has been removed.

FIG. 4 is an illustration of an embodiment of the present disclosure, for example, including an endoscopic procedure.

FIG. 5 is an additional illustration of an embodiment of FIG. 1, for example, including a spray.

FIG. 6 is an illustration of an embodiment of the present disclosure, for example, including a dressing.

FIG. 7 is an illustration of an embodiment of the present disclosure, for example, including adhesives and/or cement.

FIG. 8 is an illustration of an embodiment of the present disclosure, for example, including a cuff.

FIG. 9 is an illustration of an embodiment of the present disclosure, for example, including a balloon.

FIG. 10 is an additional illustration of an embodiment of FIG. 9.

FIG. 11 is an illustration of an embodiment of the present disclosure, for example, including a balloon positionable between tissues.

FIG. 12 is an additional illustration of the embodiments of FIG. 11.

FIG. 13 is an illustration of an embodiment of the present disclosure, for example, including a balloon with heating elements.

FIG. 14 is an illustration of an embodiment of the present disclosure, for example, including a balloon with a heat or cold pack and/or a component.

FIG. 15 is an illustration of an embodiment of the present disclosure, for example, including a balloon with a channel and/or an inlet.

FIG. 16 is an illustration of an embodiment of the present disclosure, for example, including a tool.

FIG. 17 is an illustration of an embodiment of the present disclosure, for example, including an implant.

FIG. 18 is an illustration of an embodiment of the present disclosure, for example, including an implant with an inlet, outlet, and/or channel.

FIG. 19 is an illustration of an embodiment of the present disclosure, for example, including an implant with a heating element.

FIG. 20 is an illustration of an embodiment of the present disclosure, for example, including a spinal procedure.

FIG. 21 is an illustration of an embodiment of the present disclosure, for example, an implant including micro capsules.

FIG. 22 is an additional illustration of an embodiment of FIG. 21.

FIG. 23 is an illustration of an embodiment of the present disclosure, for example, an implant including a spacer and/or sponge.

Specific features of various embodiments may be shown in some drawings and not in others, but this is for convenience only. Any feature in any drawing may be referenced and/or claimed in combination with any feature of any other drawing.

### DETAILED DESCRIPTION OF THE INVENTION

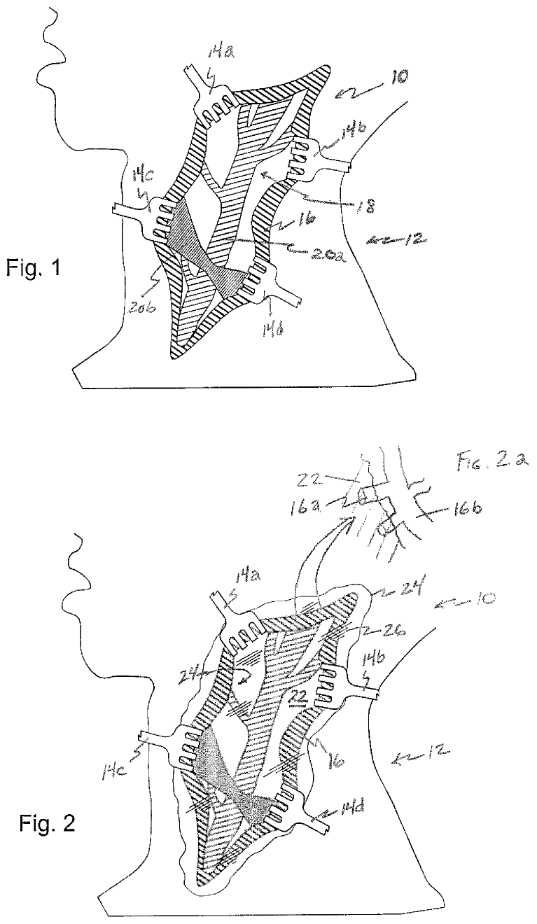

Referring now to the figures, in which like reference numerals refer to like elements, FIG. 1 illustrates a surgical operating field 10, in this illustration in the neck 12 of a patient, however in accordance with the invention, the surgical field could be anywhere in the body. Retractors 14a-d contact cut skin tissue 16, and maintain tissue 16 apart, creating operating field 10. As a result, an area of surgical interest 18 is created, containing one or more anatomical elements 20a-b upon which a surgical procedure is to be carried out. With reference now to FIG. 2, in accordance with the invention, a surgical field is filled with a viscous substance 22, illustrated by boundary line 24, hatched reflection lines 26, and the blurring or optically less clear view of operating field 10. Viscous substance 22 includes a gelatin or a polymeric solution, which serves to retard or block the ingress of blood from blood vessels in cut skin tissue 16 and other tissue 24 within the surgical field, which may also contain cut tissue and blood vessels. With reference to FIG. 2a, viscous substance 22 surrounds and adheres to the cut ends 16a of cut blood vessels 16b. The viscous substance may be optically clear, or may introduce some optical distortion as shown in FIG. 2, however some visualization of the surgical field remains, whereupon surgery may take place through viscous substance 22, as by inserting the surgeons hands (not shown) or surgical tools into viscous substance 22, as can be seen in the prongs of retractors 14a-d, and as can be seen in FIG. 4, discussed below.

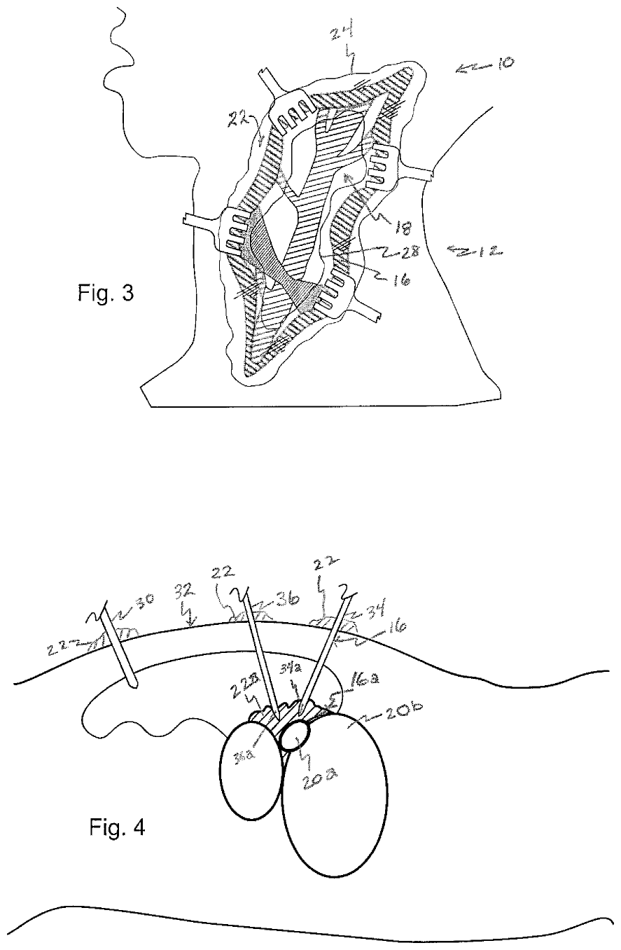

Alternatively a portion, in some cases the majority of viscous substance 22 immediately surrounding the area of interest 18 may be removed, as by manipulation and/or irrigation, so that an unobscured field of view, and an unobstructed target area are realized. In FIG. 3, boundary line 24 indicates an exterior boundary, and after a portion of viscous substance 22 has been removed, inner boundary line 28 indicates an interior boundary. Viscous substance 22, for example gelatin or polymeric gel, at the periphery of field 10 is left intact, particularly viscous substance 22 which is in direct contact with cut tissues 16,24, whereby viscous substance 22 may continue to retard entry of blood into operating field 10.

Viscosity of viscous substance 22 is advantageously in a range whereby the substance will effectively adhere to bodily tissue, without running off or dispersing during the surgical procedure, or at least, not having to be reapplied repeatedly. This represents a centipoise or cP value of at least 25. A viscosity that is too high will impose difficulties in spreading the substance on body tissue without imposing too much pressure on the tissue, typically not higher than 2,500. Values in the range of 200 to 1500 cP are advantageously employed for most body tissue. It should be understood that these values are provided as illustrative, and that features of the viscous substance as described, that is of not dispersing too quickly, or imposing too much difficulty in spreading, determine ideal viscosity for the viscous substance, based on the body tissue and application, as best determined by the surgical practitioner.

Viscous substance may be any of a wide variety of substances with the desired viscosity and biocompatibility, including gelatin, gel polymers, biocompatible lubricants, water based lubricants, silicone based lubricants, viscous degradeable polymers, and other materials described herein.

With reference to FIG. 4, the aforedescribed process in accordance with the invention may be used in both endoscopic (FIG. 4) and open incision (FIGS. 1-3) procedures. In FIG. 4, trocar introduces carbon dioxide gas into abdomen 32, facilitating the introduction and manipulation of endoscopic instruments, here laparoscopic tools 34, 36. In accordance with the invention, viscous substance 22 is placed around surgical tools 30, 34, 36 at the point of entry into abdomen 32, where skin has been cut or pierced. As the tools are inserted into the body, viscous substance 22 is driven downwards through cut skin 16 into the body, by a spreading force imparted by the surface of tools 30, 34, 36, whereby cut blood vessels inside the incision or piercing are coated with viscous substance 22 to attain the benefits as described herein. In addition, an endoscopic tool, such as tool 36, may be used to introduce viscous substance into the interior of the body, whereby it may be injected, pumped, sprayed, or brushed into contact with cut, damaged or disturbed tissue 16a. In the example shown, viscous substance 22 covers a portion of operating field 10, and the entire area of interest 18, whereby the working ends 34a, 36a of tools 34, 36 are beneath the surface of viscous substance and completely enveloped within viscous substance 22. In this manner, while there may some loss of visual clarity, the loss is offset by having the area of interest 18 substantially less obfuscated by blood.

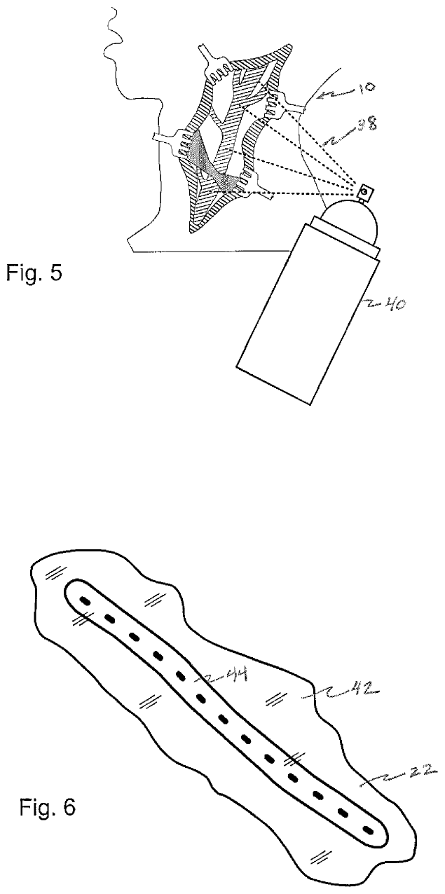

With reference to FIG. 5, in accordance with a further embodiment of the invention, surgical field 10 is sprayed with a cooling substance 38 to cool operating field 10 and cut tissue 16 sufficiently to achieve vasoconstriction, and thus reduce blood flow temporarily while the viscous substance is being applied. Cooling substance 38 may additionally be applied adjacent to the operating field, to promote vasoconstriction in surrounding tissues, and thus reduce blood flow in surgical field 10. In the example shown, a spray can 40 is illustrated, whereby substance 38 cools as it leaves can 40, due to a change of pressure. Alternatively, can 40 may be chilled prior to use. While a spray can is illustrated, it should be understood that any means of spraying is contemplated by the invention, including separate pumps, reservoirs and sprayer nozzles (not shown). Cooling substance 38 includes sterile air or water, or other substance which is biocompatible. In addition, in accordance with the invention, cooling substance may advantageously comprise viscous substance 22, although a more robust sprayer than a spray can would typically be required. Cooling substance 38 may additionally incorporate a vasoconstrictive biologic agent, such as but not limited to adenosine triphosphate, amphetamines, antihistamines, catecholamines, endothelin, ergine, methylphenidate, neuropeptide Y, norepinephrine, phenylephrine, pseudoephedrine, epinephrine, marcaine or thromboxane.

Depending upon the material used, viscous substance 22 may additionally be treated with heat, as by warm air, or cooled, as by cool spray or cool air, or alternative exposed to UV light, in order to be cured or hardened, made more durable, and caused to adhere with greater strength to cut blood vessels in cut tissue.

With reference to FIG. 6, in a further embodiment of the invention, a dressing 42, formed with UV, heat or cold setting viscous substance 22 is applied postoperatively to a surgical closure 44, providing good support to healing tissue through adhesion, and protection from infection by forming a closely conforming barrier that is impermeable to microorganisms.

The viscous substance may be formed to contain agents which aid healing or provide other therapeutic benefit, particularly substances which reduce the incidence of the formation of adhesions, which are discussed in greater detail below. Additional agents include blood clotting agents, non-steroidal anti-inflammatoires, steroidal agents, analgesics, morphine, lidocaine, other anesthetics, calcium, thrombin, hyaluronic acid, and epinephrine, and other therapeutic agents described herein.

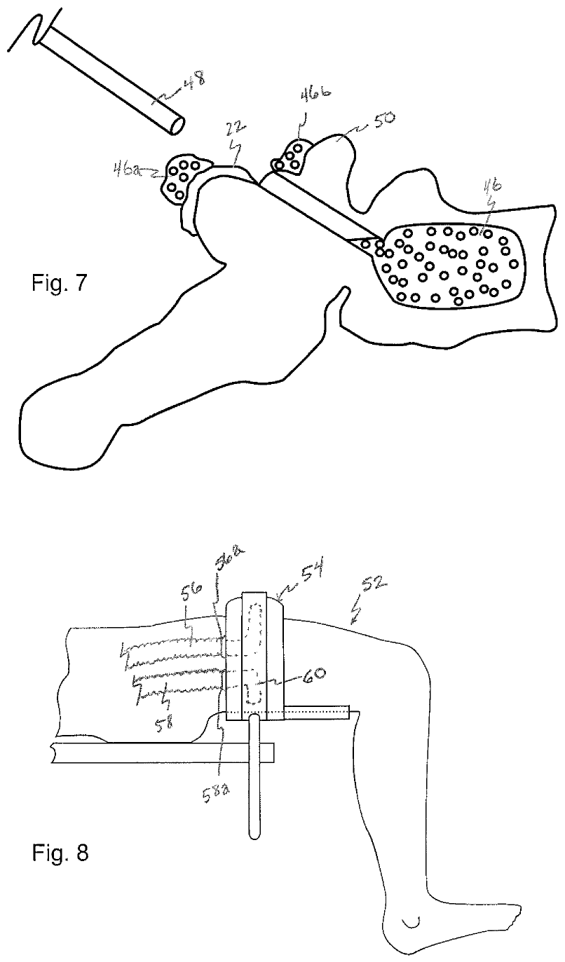

With reference to FIG. 7, in accordance with another embodiment of the invention, the introduction of a viscous substance produces a distinct advantage when working with surgical adhesives and cement 46, such as bone cement. When cement 46 is applied, as by applicator 48, it is common for a quantity of cement 46a, 46b to escape into surrounding tissue. Removal of this cement can be a time consuming process, and introduces additional risk, particularly during kyphoplasty (illustrated) and other work proximate delicate tissue. When the surrounding tissue is coated with viscous substance 22, particularly gelatin or lubricants, the removal of cement is greatly facilitated. In the example depicted, cement 46b has fallen directly onto bone 50, whereupon tools (not shown) and considerable force must be applied for removal. Should the surgical practitioner slip, grave injury may result. In contrast, cement 46a has fallen upon a substrate of viscous substance 22, and because viscous substance 22 is easily sheared, the hardened cement 46a is easily removed with greatly reduced risk to the patient

As described above, the immediate surgical field may be cooled to promote vasoconstriction, and thus reduce blood flow and blood loss. In accordance with the invention, as shown in FIG. 8, all or a portion of a limb 52 may be partially or completely surrounded by cuff 54 which is operative to squeeze the limb, and thus act as a tourniquet, while simultaneously cooling blood flowing into the limb. In the embodiment shown, cool gas or liquid, such as air or water, passes from a chiller (not shown) through inlet hose 56, through inlet 56a communicative with internal channel 60, through internal channel 60 to outlet 58a, thence through outlet hose 58 either to be recirculated or exhausted. Postoperatively, or as the surgeon deems beneficial, warmed gas or liquid may be passed through cuff 54 for therapeutic benefit, or for the comfort of the patient.

The device of FIG. 8 presents the surgeon with the opportunity to reduce the degree to which the limb must be squeezed, as the limb now additionally experiences vasoconstriction as a result of the cooling of blood vessels. Thus the trauma to muscle tissue through the physical crushing pressure and localized loss of blood flow imposed by tourniquet action is reduced.

The temperature of gas or liquid flowing through the cuff may be adjusted by either a mechanical or computer interface, or by the surgical practitioner during surgery. Temperature control in this manner is particularly useful for limb salvage surgery. The foregoing apparatus and method may advantageously be combined with epinephrine, marcain, or any of the other vasoconstrictive agents mentioned herein.

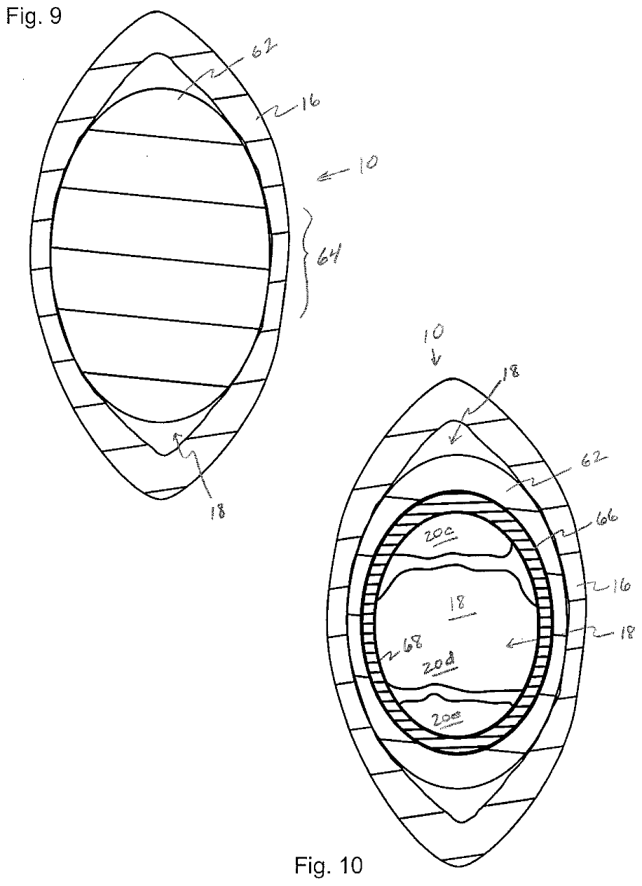

In accordance with another embodiment of the invention, and with reference to FIGS. 9 and 10, a balloon 62 is placed within surgical field 10, operative to apply pressure to bleeding tissue. Balloon 62 is formed of any biocompatible elastomeric polymer, such as rubber, latex, and synthetic rubber compounds. Balloon 62 is inflated either before or after placement within the surgical field. Where the balloon is inserted before inflation, inflation pressure is advantageously used to distract, retract, or otherwise displace tissue. In the example illustrated, cut skin 16 and other bodily tissue is pressed, for example at region of greatest pressure 64. The balloon may be sized to span the entire surgical field, whereby pressure is applied to at least two sides of the field, as is illustrated in FIG. 9. Alternatively, as shown in FIG. 11, balloon 80 may be wedged between tissue within the field, such as bone 82 or soft tissue, and bleeding tissue 16, 24. In addition, multiple balloons may be used.

In another embodiment of the invention, and with particular reference to FIG. 10, balloon 62 is caused to harden after inflation, whereupon portions of the balloon which are not engaged with bleeding tissue 16, 24, and which are not needed for support, are excised. In the illustration, a portion represented by oval 66 has been removed from the exterior portion of the balloon, after which, a portion illustrated by oval 68 is removed, whereby the surgical area of interest 18 is revealed, exposing anatomical elements 20c-e. Where it is desired to leave the remaining balloon portion within the body for a period of time, balloon 60 may advantageously be fabricated with a biodegradable material. Hardening is accomplished by coating the balloon with viscous substance 22, such as a gelatin or gel polymer, which is set or cured by cooling, heating, or exposure to UV light, as described herein. In this manner, viscous substance 22 is disposed proximate the bleeding tissue, and imparts the blood loss reduction benefits, and other therapeutic benefits, described herein.

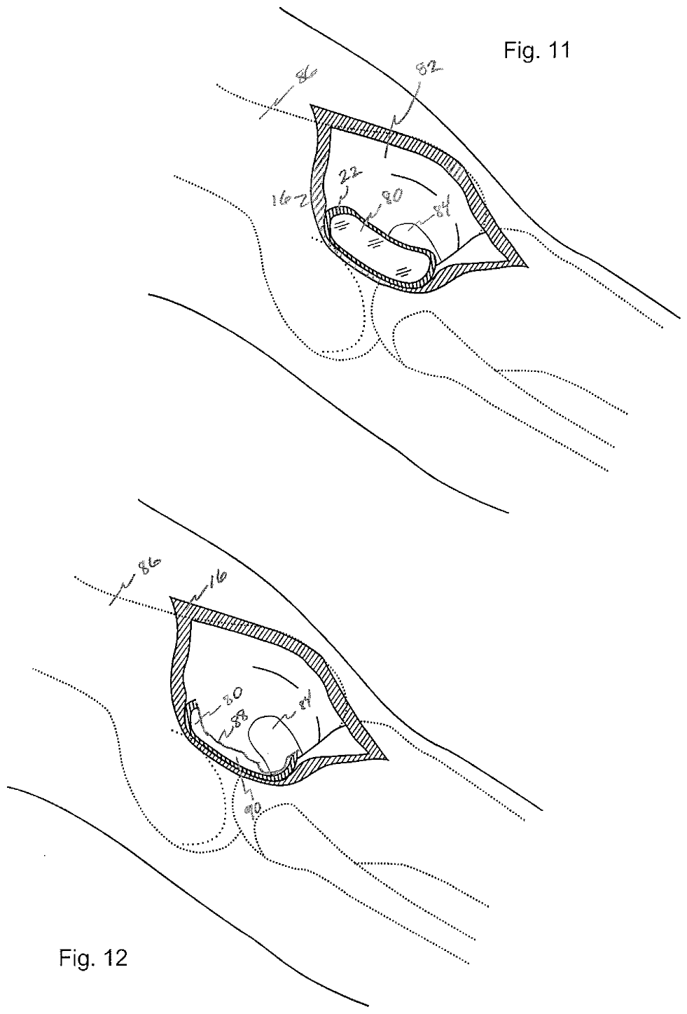

Referring now to FIGS. 11 and 12, as described above, balloon 80 is sized and shaped to advantageously fit in a part of the space within surgical field 10. In the example shown, the balloon is pressed between trochlear groove 84 of femur 86 and cut tissue 16, obscuring a portion of trochlear groove 84. Upon hardening, a portion of balloon 80 is cut at 88 to expose an additional area of trochlear groove 84. Balloon 80 remains in position against cut tissue 16 through adhesion of viscous substance 22 to cut tissue 16, 24, as well as by support from remaining hardened balloon portion 90.

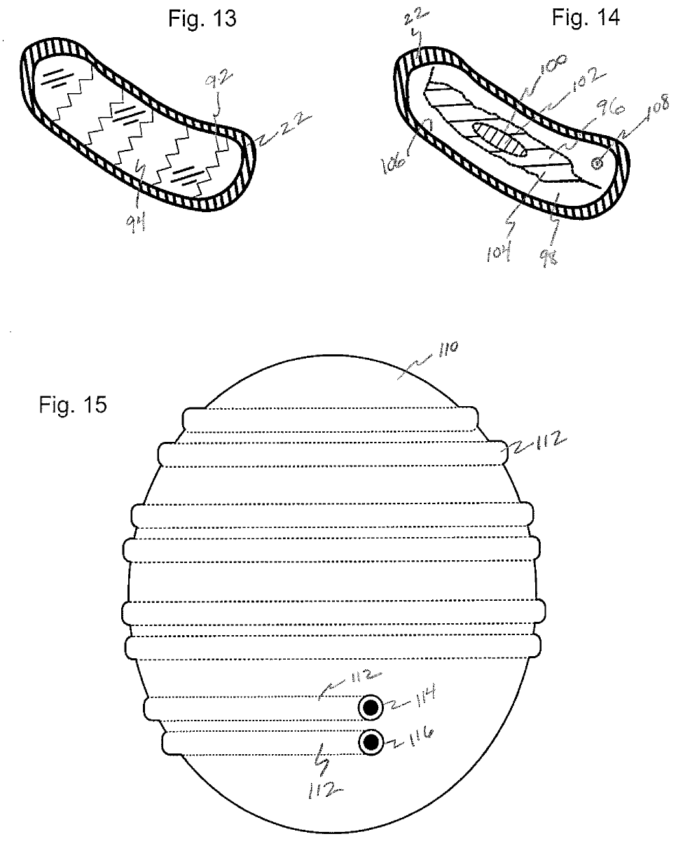

With reference to FIG. 13, in one embodiment, heating elements 92 are disposed on or within balloon 94. Elements 92 may comprise metallic or ferrous material that is heated by radiofrequency energy (RF) during and or after surgery to produce heat by induction. Alternatively, elements 92 may be connected to a source of electricity, particularly during surgery, and caused to generate heat through electrical resistance or other known means.

Alternatively, heat or cold is created by disposing chemical heat or cold packs 96 within balloon 98. Packs 96 designed to generate heat or cold upon snapping or breaking a barrier or enclosed container 100 between separated chemical components, such as components 102, 104, as known in the art, are conveniently deployed within the balloon for this purpose. To produce cold, an endothermic reaction takes place between chemical components 102, 104, and an exothermic reaction between components 102, 104 produces heat. Balloon 98 may be filled with a liquid or gas, including water or air, selected for its ability to transmit the temperature change produced by pack 96 to the surface 106 of the balloon, and to generate an even temperature on the surface 106 of balloon 98.

Similarly, component 102 may be contained within balloon 98, itself as opposed to pack 96. In this manner, container 100 is disposed within balloon 98, and is broken to produce the temperature change reaction. Container 100 may alternatively be replaced by a wall or other barrier formed within balloon 98. Further, component 104 may be injected into a balloon 98 which contains component 102; in an amount calculated to produce the appropriate amount of temperature change.

In accordance with another embodiment of the invention, as can be seen in FIG. 15, balloons 110 are provided with at least one channel 112 for conducting hot or cold flowable materials, such as liquids or gases. Flowable material of desired temperature enters inlet 114, and passes through channel 112, changing the temperature of the surface of balloon 110, and eventually exiting at outlet 116. Flowable material exiting outlet 116 may be discarded, or may be recirculated, as known in the art.

Additionally, balloons in accordance with the invention, including balloons 62, 80, 94, 98, 110 may be inflated and deflated during the surgical procedure, as by passage of a liquid or a gas through a valve 108, in order to gain access to different areas of surgical field 10, or to restore compression to bleeding tissue 16, 24. The compressive force is adjusted by varying the internal balloon pressure, or the force with which the balloon is wedged within the surgical field. In endoscopic procedures, balloons are inserted in a deflated state, as by passage through tool 30 or 36, and inflated once positioned. Advantageously, inflation pressure is only slightly higher than capillary pressure, whereby any burden on contacted tissue is minimized.

Balloons may be inflated with a gas or a liquid. Where the balloon is to be cut open, or is vulnerable to being pierced or broken, a biocompatible material, such as filtered air or sterile water, is of benefit.

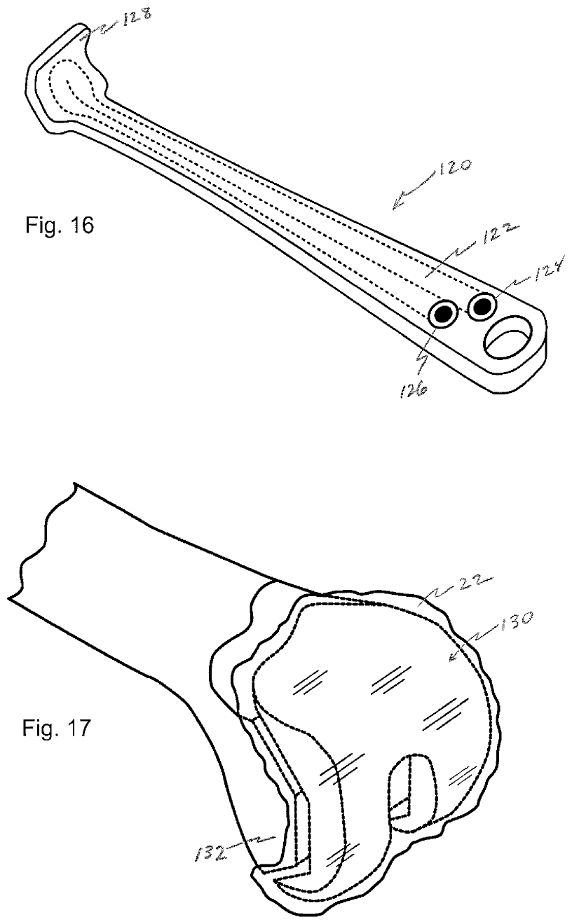

In accordance with a further embodiment of the invention, and with reference to FIG. 16, retractor 120 and other tools used within the surgical field are advantageously heated or cooled. As tools are commonly fabricated using metal, such tools may be heated or cooled prior to use. Alternatively, tools in accordance with the invention, for example retractor 120 having a channel 122 for the conduction of heated or cooled flowable material, such as a liquid or gas, are advantageously deployed. Cooled tools contribute to vasoconstriction, and may additionally be coated with gelatin or polymer gels, with attendant benefits, as described above. Tools in accordance with the invention may additionally be heated by RF radiation or heating elements, as described with respect to balloons 94, 98 of FIGS. 13 and 14.

In the example shown in FIG. 16, flowable material enters inlet 124, and travels through channel 122, adjusting the temperature of retractor 120, including tissue contacting portion 128 by conduction, eventually exiting at outlet 126.

As described for balloons and tools, above, an implant may similarly be heated or cooled, as well as coated with viscous substance 22, as described above. With reference to FIG. 17, implant 130, in this example a knee implant to be attached to resected femur 132, is coated with viscous substance 22. In accordance with the invention, the formation of postoperative adhesions is reduced due to the viscosity of viscous substance 22, and to its role as a barrier between disturbed or damaged tissue. Further, a substance known to reduce the formation of adhesions, including synovial fluid, cerebrospinal fluid, hyaluronic acid, or other materials described herein, is admixed into viscous substance 22, whereby a dual or synergistic substance anti-adhesion effect is obtained.

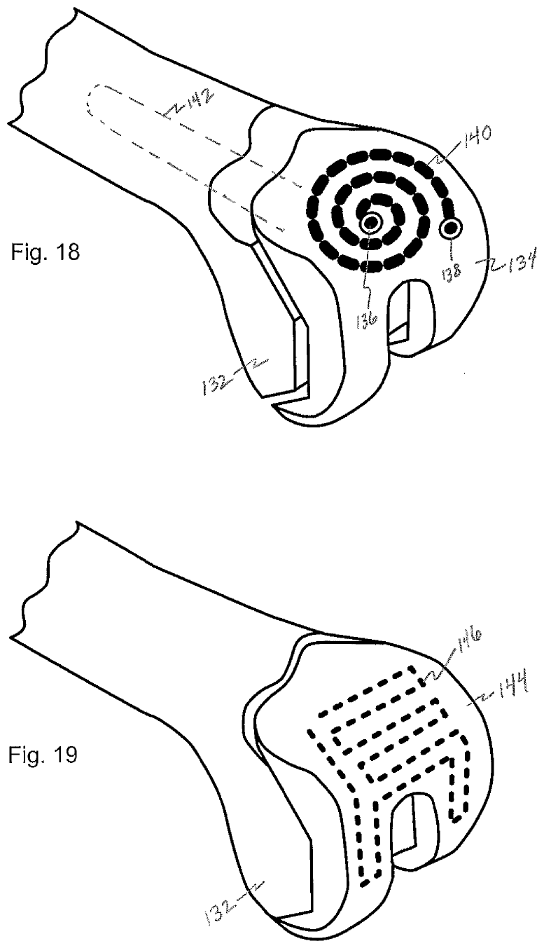

With reference to FIG. 18, implant 134 is advantageously provided with an inlet 136, outlet 138, and channel 140 for cooling or heating, as described herein. In addition, intramedullary rod 142 connected to implant 134 includes a segment of channel 140 (not shown), whereby temperature adjusted flowable material, advantageously cooled material, passes down the interior of intramedullary rod 142, thus cooling the interior of femur 132, and thereby reducing blood loss through vasoconstriction, and/or increasing the viscosity of blood through cooling.

In addition, with reference to FIG. 19, implant 144 is provided with heating element 146, controllable during surgery, or operative once an implant is secured and scaled within the body. After surgery, heat is advantageously introduced to reduce postoperative pain, and to accelerate healing. Heating may be accomplished by dielectric or induction heating, or other means not requiring an electrical connection, as described herein.

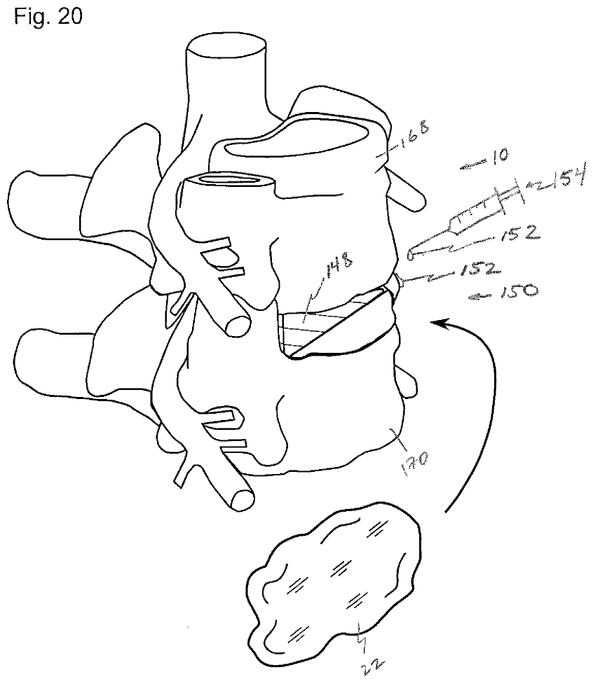

With reference to FIG. 20, a spinal implant 148 is implanted within disc space 150. Implant 148 may be provided with heating and cooling as described elsewhere herein for implants. In addition, implant 148 may be coated with viscous substance 22, as described above, to provide the therapeutic benefits described herein. Viscous substance 22 is additionally applied to cover a portion of implant 148 exposed to overlying tissue (not shown), which may have been damaged or disturbed during surgery. Further, tissue adjacent the implant has been damaged or disturbed. It has been found that adhesion formation is particularly a problem where separate tissue areas which have been disturbed or damaged, as by being abraded or cut, come into contact during healing, wherein adhesions form between the separate tissue areas. In accordance with the invention, viscous substance 22 interposed between separate tissue areas, reduces the formation of adhesions.

A medical implant in accordance with the invention can be fabricated, for example, with biodegradable polymers, cellular based materials, or other biodegradable material. The implant may additionally include a plurality of layers, each including biologic agents as described herein. Each of the multiple layers may contain the same biological agent, or medicinal agents. A treatment protocol may require that different dosages of the medicinal agent or different composition of the medicinal agent be released at different times during the treatment protocol, an immediate release vs. a delayed/retarded release. Microcapsules containing the agent or medicament are additionally contemplated, either forming one or more layers, or forming the entire implant. Implants advantageously include bone spacers or other bone implants, where the formation of adhesions can be particularly problematic. The implant can additionally be located in any other joint of the body not discussed herein, including the foot, ankle, hip, shoulder, elbow, wrist and hand.

In accordance with another embodiment of the invention, the devices and methods described above may be combined with increasing the atmospheric pressure in the operating room, in the patient, or within the surgical field, thereby further reducing blood loss.

In yet another embodiment of the invention, the various coatings of viscous substance 22 are electrically charged to cause vasoconstriction, and/or to create a diffuse cauterization of the bleeding portions 16, 24 of the surgical field 10.

In a further embodiment of the invention, blood clotting or coagulation products are admixed into the coating of viscous substance 22, whereby the viscous substance effectively maintains the products in close conformity to the cut ends 16a of cut blood vessels 16b.

In accordance with yet another embodiment of the invention, a combination of therapeutic substances may be administered to the patient, cooperative with the devices and methods of the invention, to increase the overall efficacy of the procedure. These may be delivered before or after surgery, and may be timed release. Additionally, any implanted device, balloon or other implant, in accordance with the invention, may be formulated to be drug eluting, either through incorporation into the gelatin or gel matrix which coats the device, as described above, or by formulating the device to contain therapeutic substances which are released by known means, including biodegradation.

In accordance with a further embodiment of the invention, system or local pH is made more alkaline, in order to decrease the caustic effect of bleeding, thus protecting soft tissue and decreasing pain.

As discussed briefly above, and will be more particularly described below, the present invention provides a method of using a biologic agent to inhibit scar formation, in particular, surgical adhesions and exogenous bone formation. The biologic agent is biodegradable and is thus reabsorbed over a period of time. The biologic agent can be used to prevent or inhibit the formation of adhesions in an animal following any type of surgery or trauma, by applying an effective amount of the biologic agent to a wound site.

The wound site refers to a site of tissue that has been injured in any manner, e.g., through surgery, contusion, abrasion, and so forth, and also refers to tissues or organs that arc adjacent to the injured tissue. For example, the biologic agent may be used to prevent or inhibit adhesions that form in relation to intestinal surgery, e.g., bowel resection, hernia repair, etc., which may cause obstruction of the intestine. The biologic agent may also prevent or inhibit adhesions or exogenous bone formation that can form near a bone fracture site, joint repair or replacement site, the formation of which may reduce or hinder the normal movement of the area of repair by restricting the natural movement of tendons over adjacent bone.

The biologic agent may be included with a composition within a carrier material, e.g., water, gel, or a nonaqueous solvent. To aid in healing, the composition can additionally include a medicinal agent. Exemplary medicinal agents include drugs, enzymes, proteins, hormones, peptides, glycoproteins, or diagnostic agents such as releasable dyes which may have no biological activity per se.

Examples of classes of medicinal agents that can be used include antimicrobials, analgesics, antipyretics, anesthetics, antiepileptics, antihistamines, anti-inflammatories, anti-clotting agents, bone morphogenic proteins, cardiovascular drug, diagnostic agents, sympathomimetics, cholinomimetics, anti-muscarinics, antispasmodics, hormones, growth factors, muscle relaxants, adrenergic neuron blocks, anti-neoplastics, immunosuppressants, gastrointestinal drugs, diuretics, steroids and enzymes. It is also intended that combinations of medicinal agents can be used.

In addition to or as an alternative to, the medicinal agent may be a therapeutic agent. Examples of such agents include, but are not limited to, hormones, cells, fetal cells, stem cells, bone morphogenic proteins (BMPs), enzymes, proteins, RNA, germicides, gene therapy substances, cell therapy substances, viruses, etc.

In one embodiment of the invention, the biologic agent is synovial fluid. The synovial fluid can be harvested from the patient prior to or during the surgical procedure by known techniques. Alternatively, the synovial fluid can be harvested from a donor.

Alternatively, the biologic agent is cerebrospinal fluid. The cerebrospinal fluid can be harvested from the patient prior or during the procedure by known techniques. Alternatively, the cerebrospinal fluid can be harvested from a donor.

Referring to FIG. 20, an exemplary surgical site or field 10 is provided. The surgical field 10 can be, as examples, intestinal, cardiac, or joint sites. During and or after the surgical procedure, the biologic agent 152 is applied to the surgical field 10 by any convenient mode 154, including admixed into viscous substance 22 as described above.

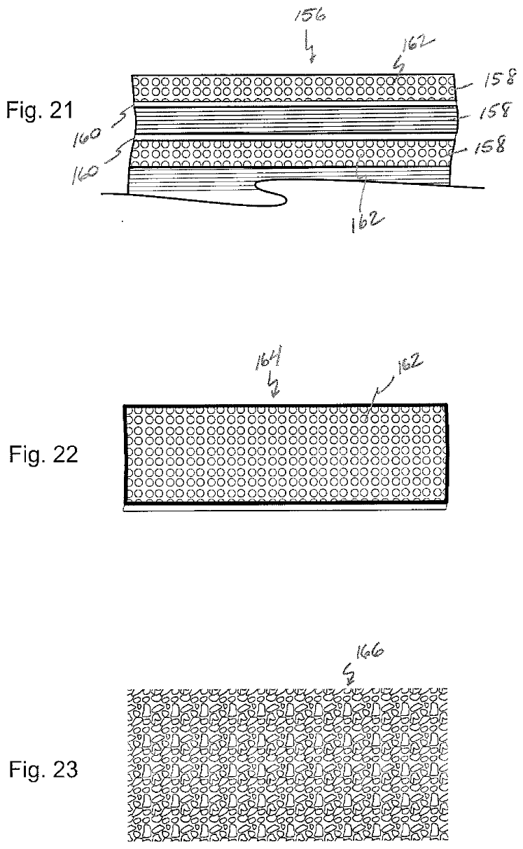

Referring to FIG. 21, the biodegradable implant 156 may be made up of a plurality of layers or sections 158, each including the biologic agent 152. The biologic agent 152 is released as the layers 158 of the biodegradable implant degrade. The degradation rate of the biodegradable implant 156 can be controlled by the ratio of PLA to PGA, or by the thickness or density of the layers 158, or interposed layers 160.

Each of the multiple layers may contain the same biological agent 152 as well and a medicinal agent. The medicinal agents (and/or the composition of the agents) in each of the multiple layers may be the same or different. A treatment protocol may require that different dosages of the medicinal agent or different composition of the medicinal agent be released at different times during the treatment protocol, an immediate release vs. a delayed/retarded release. The multiple-layers, each containing different dosages of the medicinal agents or different compositions of the medicinal agents, allow for the controllable release of the differing medicinal agents during the protocol.

Referring again to FIG. 21, the medical implant 156 includes at least one layer of micro capsules 162. The biologic agent 152 is contained within the micro capsule 162. The micro capsules 162 may be bonded to the medical implant 156 with a biodegradable agent, such that as the biodegradable agent degrades, micro capsules 162 are released. Similarly, the micro capsules 162 may be made of a biodegradable material, such that as the micro capsules 162 degrade, the biologic agent 152 will be released.

Alternatively, as can be seen in FIG. 22, the medical implant 164 may be made entirely of micro capsules 162 bonded together. The bonded microcapsule 162 can be appropriately shaped and sized depending on the intended area of use. The micro capsules 162 may be bonded together with a biodegradable agent, such that as the biodegradable agent degrades the micro capsules 162 are released. Similarly, the micro capsules 162 may be made of a biodegradable material, such that as the micro capsules 162 degrade the biologic agent 152 will be released.

Referring to FIG. 23 the medical implant 166 is a spacer or sponge. The biologic agent 152 is incorporated in the medical implant 164, for insertion into the surgical site. The biologic agent 152 seeps from the medical implant 164 to the surrounding tissue. Additionally, the biologic agent 152 can be applied to the surrounding tissue as described above.

The medical implant 166 can be a biodegradable implant. The biodegradable implant 166 hydrophilically reacts to release the biologic agent 152. The biodegradable implant 166 is made of a biodegradable polymer, polyactic acid (“PLA”), polyglycolic acid (“PGA”), and copolymers thereof collagen, cellulose, fibrin, autograft, allograft, or other cellular based compounds. The biologic agent 152 may be affixed to the biodegradable implant by coating, mixing, or bonding techniques.

Referring back to FIG. 20, in an alternative embodiment, implant 148 is a joint spacer, for changing the spatial relationship between first and second bones 168, 170, and incorporates biologic agent 152 as described above. The medical implant 148 includes a body configured and dimensioned for insertion into a joint 150 located between the first and second bones 168, 170. As discussed above the biologic agent 152 is incorporated into or coated on the implant 148. The biological agent 152 inhibits the formation of adhesion, scar tissue, or exogenous bone that would limit the movement of the first and second bones 168, 170, or that might otherwise cause pain or discomfort. Additionally, the biologic agent 152 can be applied to the surrounding tissue as described above.

Implant 148 can be a temporary spacer, left in position for a set time period, upon expiration of which the implant 148 is removed and/or replaced. For example, in younger patients, not suitable candidates for spinal fusion, implant 148 is inserted between the effected vertebrae, to stabilize the spinal area for a period of time. At the expiration of the time period, patient is evaluated. The implant 148 is then removed and, if required, replaced.

Alternatively, the implant 148 is made of a biodegradable material. The biologic agent 152 is incorporated in biodegradable implant 148, for insertion in between the vertebrae. The biologic agent 152 seeps from the biodegradable interveltebral spacer 22 to the surrounding tissue. Additionally, the biologic agent 152 can be applied to the surrounding tissue as described above.

In another embodiment in accordance with the invention, implant 148 hydrophilically reacts to release the biologic agent 152. Implant 148 is made of a biodegradable polymer, polyactic acid (“PLA”), polyglycolic acid (“PGA”) and copolymers thereof collagen, cellulose, fibrin, autograph, allograph, or other cellular based compounds. The biologic agent 152 may be affixed to the biodegradable implant by coating, mixing, or bonding the biologic agent to the biodegradable intervertebral spacer 22.

Referring again to FIG. 21, the implant 148 may be made of a plurality of layers or sections 158, each including the biologic agent 152. The biologic agent 152 is released as the layers 158 of the biodegradable implant degrade. The degradation rate of the biodegradable implant can be controlled by the ratio of PLA to PGA, or by the thickness or density of the layers, as described above.

All references cited herein are expressly incorporated by reference in their entirety.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention.

1-23. (canceled)

24. A system for treatment comprising:
a medical implant configured to be implanted into a bone;
microcaspsules comprised of a biodegradable material and bonded to the medical implant with a biodegradable agent, the microcapsules forming a layer covering the exterior surface of the medical implant; and
at least one therapeutic agent contained within the microcapsules, the therapeutic agent configured to be released into a treatment site as the microcapsules are released from the medical implant as the biodegradable agent degrades and as the microcapsules degrade.
